(19) United States
(12) Patent Application Publication
(10) Pub. No.: US 2014/0200380 A1
(43) Pub. Date: Jul. 17, 2014

(54) Process for Preparing 1,3-Butadiene from N-Butenes by Oxidative Dehydrogenation

(71) Applicant: BASF SE, Ludwigshafen (DE)

(72) Inventors: Jan Pablo Josch, Neustadt (DE); Philipp Grüne, Mannheim (DE); Christian Walsdorff, Ludwigshafen (DE); Oliver Hammen, Wintersheim (DE); Ragavendra Prasad Balegedde Ramachandran, Ludwigshafen (DE)

(73) Assignee: BASF SE, Ludwigshafen (DE)

(21) Appl. No.: 14/154,558

(22) Filed: Jan. 14, 2014

Related U.S. Application Data
(60) Provisional application No. 61/752,543, filed on Jan. 15, 2013.

(51) Int. Cl. C07C 7/05; C07C 5/48

(52) U.S. Cl. CPC C07C 7/05; C07C 5/48
USPC 585/621

(57) ABSTRACT

The invention relates to a process for preparing butadiene from n-butenes, which comprises the steps:
A) provision of a feed gas stream a comprising n-butenes;
B) introduction of the feed gas stream a comprising n-butenes and an oxygen-comprising gas into at least one oxidative dehydrogenation zone and oxidative dehydrogenation of n-butenes to butadiene, giving a product gas stream b comprising butadiene, unreacted n-butenes, water vapor, oxygen, low-boiling hydrocarbons, high-boiling secondary components, possibly carbon oxides and possibly inert gases;
Ca) cooling of the product gas stream b by contacting with an organic solvent as coolant,
Cb) compression of the product gas stream b in at least one compression stage, giving at least one aqueous condensate stream c1 and a gas stream c2 comprising butadiene, n-butenes, water vapor, oxygen, low-boiling hydrocarbons, possibly carbon oxides and possibly inert gases;
D) separation of incondensable and low-boiling gas constituents comprising oxygen, low-boiling hydrocarbons, possibly carbon oxides and possibly inert gases as gas stream d2 from the gas stream c2 by absorption of the C4-hydrocarbons comprising butadiene and n-butenes in an absorption medium, giving an absorption medium stream loaded with C4 -hydrocarbons and the gas stream d2, and subsequent desorption of the C4-hydrocarbons from the loaded absorption medium stream to give a C4-product gas stream d1,
E) separation of the C₄ product stream d1 by extractive distillation using a solvent selective for butadiene into a stream e1 comprising butadiene and the selective solvent and a stream e2 comprising n-butenes;
F) distillation of the stream f2 comprising butadiene and the selective solvent to give a stream g1 consisting essentially of the selective solvent and a stream g2 comprising butadiene.

### CROSS-REFERENCE TO RELATED APPLICATIONS

This application claims benefit (under 35 USC 119(e)) of U.S. Provisional Application No. 61/752,543, filed Jan. 15, 2013, which is incorporated by reference.

### FIELD OF THE INVENTION

The invention relates to a process for preparing 1,3-butadiene from n-butenes by oxidative dehydrogenation (ODH).

### BACKGROUND OF THE INVENTION

Butadiene is an important basic chemical and is used, for example, for preparing synthetic rubbers (butadiene homopolymers, styrene-butadiene rubber or nitrile rubber) or for preparing thermoplastic terpolymers (acrylonitrile-butadiene-styrene copolymers). Butadiene is also converted into sulfolane, chloroprene and 1,4-hexamethylenediamine (via 1,4-dichlorobutene and adiponitrile). Furthermore, dimerization of butadiene makes it possible to produce vinylcyclohexene which can be dehydrogenated to form styrene.

Butadiene can be prepared by thermal dissociation (steam cracking) of saturated hydrocarbons, usually using naphtha as raw material. The steam cracking of naphtha gives a hydrocarbon mixture composed of methane, ethane, ethene, acetylene, propane, propene, propyne, allene, butanes, butenes, butadiene, butynes, methylallene, C₅-hydrocarbons and higher hydrocarbons.

Butadiene can also be obtained by oxidative dehydrogenation of n-butenes (1-butene and/or 2-butene). As starting gas mixture for the oxidative dehydrogenation (oxydehydrogenation, ODH) of n-butenes to butadiene, it is possible to use any mixture comprising n-butenes. For example, it is possible to use a fraction which comprises n-butenes (1-butene and/or 2-butene) as main constituent and has been obtained from the C4 fraction from a naphtha cracker by removal of butadiene and isobutene. Furthermore, it is also possible to use, as starting gas, gas mixtures which comprise 1-butene, cis-2-butene, trans-2-butene or mixtures thereof and have been obtained by dimerization of ethylene. Furthermore, it is possible to use gas mixtures which comprise n-butenes and have been obtained by fluid catalytic cracking (FCC) as starting gas.

Processes for the oxidative dehydrogenation of butenes to butadiene are known per se.

For example, US 2012/0130137 A1 describes a process of this type using catalysts comprising the oxides of molybdenum, bismuth and generally further metals. To maintain the activity of such catalysts for the oxidative dehydrogenation in the long term, a critical minimum oxygen partial pressure in the gas atmosphere is necessary in order to avoid substantial reduction and thus a decrease in performance of the catalysts. For this reason, it is generally also not possible to work with a stoichiometric input of oxygen or complete oxygen conversion in the oxydehydrogenation reactor (ODH reactor). US 2012/0130137 describes, for example, an oxygen content of 2.5-8% by volume in the starting gas.

The necessity of an excess of oxygen for such catalyst systems is generally known and established in the process conditions when using such catalysts. As representative references, mention may be made of the relatively recent work by Jung et al. (Catal. Surv. Asia 2009, 13, 78-93; DOI 10.1007/s10563-009-9069-5 and Applied Catalysis A: General 2007, 317, 244-249; DOI 10.1016/j.apcata.2006.10.021).

However, the presence of oxygen in addition to butadiene after the ODH reactor stage is associated with risks in the work-up part of such processes operated using an excess of oxygen. Particularly in the liquid phase, the formation and accumulation of organic peroxides has to be examined. These risks are discussed, for example by D. S. Alexander (Industrial and Engineering Chemistry 1959, 51, 733-738).

JP-A 2011-006381 by Mitsubishi addresses the risk of peroxide formation in the work-up part of a process for preparing conjugated alkadienes. The solution described is the addition of polymerization inhibitors to the absorption solutions for the process gases and setting of a maximum peroxide content of 100 ppm by weight by heating the absorption solutions. However, nothing is said about avoiding or controlling peroxides in upstream process steps. The cooling step for the ODH reactor output using a water quench is particularly critical. Organic peroxides formed are sparingly soluble in water, so that they precipitate and can accumulate in solid or liquid form in the apparatus, instead of being discharged with the aqueous purge stream. At the same time, the temperature of the water quench is not high enough for a sufficiently substantial and continual degradation of the peroxides formed to be able to be assumed.

In the catalytic oxidative dehydrogenation, high-boiling secondary components such as maleic anhydride, phthalic anhydride, benzaldehyde, benzoic acid, ethylbenzene, styrene, fluorenone, anthraquinone and others can be formed. Such deposits can lead to blockages and to an increase in the pressure drop in the reactor or downstream of the reactor in the region of the work-up and thus lead to malfunctions in regulated operation. Deposits of the high-boiling secondary components mentioned can also impair the function of heat exchangers or damage moving apparatuses such as compressors. Steam-volatile compounds such as fluorenone can get through a quenching apparatus operated using water and deposit downstream in the gas discharge lines. There is therefore in principle also the risk that solid deposits enter downstream parts of the apparatus, for example compressors, and cause damage there.

US 2012/0130137 A1 paragraph [0122] also refers to the problems of high-boiling by-products. Particular mention is made of phthalic anhydride, anthraquinone and fluorenone, which are typically present in concentrations of 0.001-0.10% by volume in the product gas. In US 2012/0130137 A1 paragraph [0124]-[0126] it is recommended that the hot reactor output gases be cooled to usually firstly 5-100° C. by direct contact with a cooling liquid (quenching tower). Cooling liquids mentioned are water or aqueous alkali solutions. Explicit mention is made of the problems of blockages in the quench caused by high boilers from the product gas or by polymerization products of high-boiling secondary products from the product gas, which is why it is said to be advantageous for high-boiling secondary products to be carried from the reaction section into the cooling section (quench) to the smallest extent possible.

JP-A 2011-001341 describes two-stage cooling for a process for the oxidative dehydrogenation of alkenes to conjugated alkadienes. Here, the product output gas from the oxidative dehydrogenation is firstly brought to a temperature in the range from 300 to 221° C. and then cooled further to a temperature in the range from 99 to 21° C. In paragraphs [0066] if, it is stated that heat exchangers are preferably used for setting the temperature in the range from 300 to 221° C., but part of the high boilers can also precipitate from the product gas in these heat exchangers. For this reason, occasional washing-out of the deposits from the heat exchangers by means of organic or aqueous solvents is described in JP-A 2011-001341. Solvents described by way of example are aromatic hydrocarbons such as toluene or xylene or an alkaline aqueous solvent such as an aqueous solution of sodium hydroxide. To avoid frequent shutting-down of the process in order to clean the heat exchangers, JP-A 2011-001341 describes a set-up with two heat exchangers which are arranged in parallel and are alternately operated or flushed (known as NB mode of operation).

### BRIEF DESCRIPTION OF THE DRAWINGS

FIG. 1 shows a first section of an embodiment of the process of the invention.

FIG. 2 shows a second section of an embodiment of the process of the invention.

FIG. 3 shows a third section of an embodiment of the process of the invention.

### DETAILED DESCRIPTION OF THE INVENTION

It is an object of the present invention to provide a process which remedies the abovementioned disadvantages of known processes. In particular, a process in which deposits caused by high-boiling organic secondary constituents in the apparatuses located downstream of the ODH are avoided should be provided. Furthermore, a process in which possible accumulation of organic peroxides is avoided should be provided. Furthermore, it is an object of the invention to reduce high pollution of wastewater with organic compounds in dissolved, emulsified or suspended form and also the formation of wastewater polluted with organic compounds.

The object is achieved by a process for preparing butadiene from n-butenes, which comprises the steps:
A) provision of a feed gas stream a comprising n-butenes;
B) introduction of the feed gas stream a comprising n-butenes and an oxygen-comprising gas into at least one oxidative dehydrogenation zone and oxidative dehydrogenation of n-butenes to butadiene, giving a product gas stream b comprising butadiene, unreacted n-butenes, water vapor, oxygen, low-boiling hydrocarbons, high-boiling secondary components, possibly carbon oxides and possibly inert gases;
Ca) cooling of the product gas stream b by contacting with an organic solvent;
Cb) compression of the product gas stream b in at least one compression stage, giving at least one aqueous condensate stream c1 and a gas stream c2 comprising butadiene, n-butenes, water vapor, oxygen, low-boiling hydrocarbons, possibly carbon oxides and possibly inert gases;
D) separation of incondensable and low-boiling gas constituents comprising oxygen, low-boiling hydrocarbons, possibly carbon oxides and possibly inert gases as gas stream d2 from the gas stream c2 by absorption of the C₄-hydrocarbons comprising butadiene and n-butenes in an absorption medium, giving an absorption medium stream loaded with C₄-hydrocarbons and the gas stream d2, and subsequent desorption of the C₄-hydrocarbons from the loaded absorption medium stream to give a C4-product gas stream d1;
E) separation of the C4 product stream d1 by extractive distillation using a solvent selective for butadiene into a stream e1 comprising butadiene and the selective solvent and a stream e2 comprising n-butenes;
F) distillation of the stream e1 comprising butadiene and the selective solvent to give a stream f1 consisting essentially of the selective solvent and a stream f2 comprising butadiene.

In a step A), a feed gas stream comprising n-butenes is provided.

According to the invention, an organic solvent is used in the cooling step Ca). This generally has a very much higher solvent capability for the high-boiling by-products which can lead to deposits and blockages in the ODH reactor and downstream plant components than for water or alkaline-aqueous solutions. Organic solvents preferably used as coolants are aromatic hydrocarbons, particularly preferably toluene, o-xylene, m-xylene, p-xylene or mixtures thereof.

The following embodiments are preferred or particularly preferred variants of the process of the invention:

Step Ca) is carried out in a plurality of stages in steps Ca1) to Can), preferably in two stages Ca1) and Ca2). Here, at least part of the solvent which has gone through the second stage Ca2) is particularly preferably used as coolant in the first stage Ca1).

Step Cb) generally comprises at least one compression stage Cba) and at least one cooling stage Cbb). The gas compressed in the compression stage Cba) is preferably brought into contact with a coolant in the at least one cooling stage Cbb). The coolant of cooling stage Cbb) particularly preferably comprises the same organic solvent that is used as coolant in stage Ca). In a particularly preferred variant, at least part of the coolant which has gone through the at least one cooling stage Cbb) is fed as coolant into the stage Ca).

The stage Cb) preferably comprises a plurality of compression stages Cba1) to Cban) and cooling stages Cbb1) to Cbbn), for example four compression stages Cba1) to Cba4) and four cooling stages Cbb1) to Cbb4).

Step D) preferably comprises the steps Da) to Dc):
Da) absorption of the C₄-hydrocarbons comprising butadiene and n-butenes in a high-boiling absorption medium, giving an absorption medium stream loaded with C₄-hydrocarbons and the gas stream d2,
Db) removal of oxygen from the absorption medium stream loaded with C₄-hydrocarbons from step Da) by stripping with an incondensable gas stream and
Dc) desorption of the C₄-hydrocarbons from the loaded absorption medium stream, giving a C₄-product gas stream d1 which consists essentially of C₄-hydrocarbons and comprises less than 100 ppm of oxygen.

The high-boiling absorption medium used in step Da) is preferably an aromatic hydrocarbon solvent, particularly preferably the aromatic hydrocarbon solvent used in step Ca), in particular toluene.

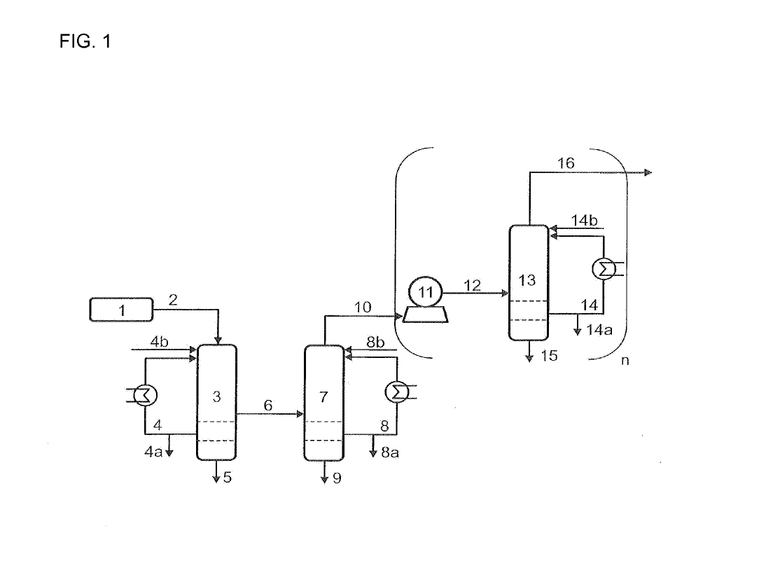

Embodiments of the process of the invention are shown in FIGS. 1-3 and are described in detail below.

As feed gas stream, it is possible to use pure n-butenes (1-butene and/or cis-/trans-2-butene) or else gas mixtures comprising butenes. Such a gas mixture can, for example, be obtained by nonoxidative dehydrogenation of n-butane. It is also possible to use a fraction which comprises n-butenes (1-butene and cis-/trans-2-butene) as main constituent and has been obtained from the C₄ fraction from naphtha cracking by removal of butadiene and isobutene. Furthermore, it is also possible to use gas mixtures which comprise pure 1-butene, cis-2-butene, trans-2-butene or mixtures thereof and have been obtained by dimerization of ethylene as starting gas. Furthermore, gas mixtures comprising n-butenes which have been obtained by fluid catalytic cracking (FCC) can be used as starting gas.

In an embodiment of the process of the invention, the starting gas mixture comprising n-butenes is obtained by nonoxidative dehydrogenation of n-butane. Coupling of a nonoxidative catalytic dehydrogenation with the oxidative dehydrogenation of the n-butenes formed enables a high yield of butadiene, based on n-butane used, to be obtained. The nonoxidative catalytic dehydrogenation of n-butane gives a gas mixture comprising butadiene, 1-butene, 2-butene and unreacted n-butane and also secondary constituents. Usual secondary constituents are hydrogen, water vapor, nitrogen, CO and CO₂, methane, ethane, ethene, propane and propene. The composition of the gas mixture leaving the first dehydrogenation zone can vary greatly as a function of the way in which the dehydrogenation is carried out. Thus, when the dehydrogenation is carried out with introduction of oxygen and additional hydrogen, the product gas mixture has a comparatively high content of water vapor and carbon oxides. In a mode of operation without introduction of oxygen, the product gas mixture from the nonoxidative dehydrogenation has a comparatively high content of hydrogen.

In step B), the feed gas stream comprising n-butenes and an oxygen-comprising gas are fed into at least one dehydrogenation zone (the ODH reactor 1) and the butenes comprised in the gas mixture are oxidatively dehydrogenated to butadiene in the presence of an oxydehydrogenation catalyst.

Catalysts suitable for the oxydehydrogenation are generally based on an Mo—Bi—O-comprising multimetal oxide system which generally additionally comprises iron. In general, the catalyst system comprises further additional components such as potassium, cesium, magnesium, zirconium, chromium, nickel, cobalt, cadmium, tin, lead, germanium, lanthanum, manganese, tungsten, phosphorus, cerium, aluminum or silicon. Iron-comprising ferrites have also been proposed as catalysts.

In a preferred embodiment, the multimetal oxide comprises cobalt and/or nickel. In a further preferred embodiment, the multimetal oxide comprises chromium. In a further preferred embodiment, the multimetal oxide comprises manganese.

Examples of Mo—Bi—Fe—O-comprising multimetal oxides are Mo—Bi—Fe—Cr—O— or Mo—Bi—Fe—Zr-β-comprising multimetal oxides. Preferred systems are, for example, described in U.S. Pat. No. 4,547,615 (Mo₁₂BiFe_{0.1}Ni₈ZrCr₃K_{0.2}Oₓ and Mo₁₂BiFe_{0.1} Ni₈AlCr₃K_{0.2}Oₓ), U.S. Pat. No. 4,424,141 (Mo₁₂BiFe₃Cu_{4.5}Ni_{2.5}P_{0.5}K_{0.1}Oₓ+SiO₂), DE-A 25 30 959 (Mo₁₂BiFe₃Cu_{4.5}Ni_{2.5}Cr_{0.5}K_{0.1}Oₓ, Mo_{13.75}BiFe₃Cu_{4.5}Ni_{2.5}Ge_{0.5}K_{0.8}Oₓ, Mo₁₂BiFe₃Co_{4.5}Ni_{2.5}Mn_{0.5}K_{0.1}Oₓ, and Mo₁₂BiFe₃Cu_{4.5}Ni_{2.5}La_{0.5}K_{0.1}Oₓ), U.S. Pat. No. 3,911,039 (Mo₁₂BiFe₃Cu_{4.5}Ni_{2.5}Sn_{0.5}K_{0.1}Oₓ), DE-A 25 30 959 and DE-A 24 47 825 (Mo₁₂BiFe₃Cu_{4.5}Ni_{2.5}W_{0.5}K_{0.1}Oₓ).

Suitable multimetal oxides and the preparation thereof are also described in U.S. Pat. No. 4,423,281 (Mo₁₂BiNi₈Pb_{0.5}Cr₃K_{0.2}Oₓ and Mo₁₂BibNi₇Al₃Cr_{0.5}K_{0.5}Oₓ), U.S. Pat. No. 4,336,409 (Mo₁₂BiNi₆Cd₂Cr₃P_{0.5}Oₓ), DE-A 26 00 128 (Mo₁₂BiNia₅Cr₃P_{0.5}Mg_{7.5}K_{0.1}Oₓ+SiO₂) and DE-A 24 40 329 (Mo₁₂BiCO_{4.5}Ni_{2.5}Cr₃P_{0.5}K_{0.1}Oₓ).

Particularly preferred catalytically active multimetal oxides comprising molybdenum and at least one further metal have the general formula (Ia):

Mo₁₂BiₐFe_{b}Co_{c}Ni_{d}CrₑX¹_{f}X²_{g}O_{y} (Ia),

where
X¹=Si, Mn and/or Al,
X²=Li, Na, K, Cs and/or Rb,
0.2≦a≦1,
0.5≦b≦10,
0≦c≦10,
0≦d≦10,
2≦c+d≦10,
0≦e≦2,
0≦f≦10,
0≦g≦0.5,
y=a number which is determined by the valence and abundance of the elements other than oxygen in (Ia) so as to achieve charging neutrality.

Preference is given to catalysts whose catalytically active oxide composition comprises only Co (d=0) from among the two metals Co and Ni X¹ is preferably Si and/or Mn and X² is preferably K, Na and/or Cs, particularly preferably X²=K.

The gas comprising molecular oxygen generally comprises more than 10% by volume, preferably more than 15% by volume and even more preferably more than 20% by volume, of molecular oxygen. It is preferably air. The upper limit to the content of molecular oxygen is generally 50% by volume or less, preferably 30% by volume or less and even more preferably 25% by volume or less. In addition, any inert gases can be comprised in the gas comprising molecular oxygen. Possible inert gases are nitrogen, argon, neon, helium, CO, CO₂ and water. The amount of inert gases is in the case of nitrogen generally 90% by volume or less, preferably 85% by volume or less and even more preferably 80% by volume or less. In the case of constituents other than nitrogen, it is generally 10% by volume or less, preferably 1% by volume or less.

To carry out the oxidative dehydrogenation at full conversion of n-butenes, preference is given to a gas mixture having a molar oxygen:n-butenes ratio of at least 0.5. The oxidative dehydrogenation is preferably carried out at an oxygen:n-butenes ratio of from 0.55 to 10. To set this value, the starting gas can be mixed with oxygen or an oxygen-comprising gas, for example air, and optionally additional inert gas or water vapor. The oxygen-comprising gas mixture obtained is then fed to the oxydehydrogenation.

The reaction temperature of the oxydehydrogenation is generally controlled by means of a heat transfer medium which is present around the reaction tubes. Possible liquid heat transfer media of this type are, for example, melts of salts such as potassium nitrate, potassium nitrite, sodium nitrite and/or sodium nitrate and also melts of metals such as sodium, mercury and alloys of various metals. However, ionic liquids or heat transfer oils can also be used. The temperature of the heat transfer medium is in the range from 220 to 490° C., preferably in the range from 300 to 450° C. and particularly preferably in the range from 350 to 420° C.

Owing to the exothermic nature of the reaction which occurs, the temperature in particular sections of the interior of the reactor can be higher than that of the heat transfer medium during the reaction and a hot spot is formed. The position and magnitude of the hot spot is determined by the reaction conditions, but can also be regulated by the dilution ratio of the catalyst bed or the flow of mixed gas through the bed. The difference between hot spot temperature and the temperature of the heat transfer medium is generally in the range 1-150° C., preferably 10-100° C. and particularly preferably 20-80° C. The temperature at the end of the catalyst bed is generally 0-100° C. above, preferably 0.1-50° C. above, particularly preferably 1-25° C. above, the temperature of the heat transfer medium.

The oxydehydrogenation can be carried out in all fixed-bed reactors known from the prior art, for example in tray ovens, in a fixed-bed tube reactor or shell-and-tube reactor or in a plate heat exchanger. A shell-and-tube reactor is preferred.

The oxidative dehydrogenation is preferably carried out in fixed-bed tube reactors or fixed-bed shell-and-tube reactors. The reaction tubes are (like the other elements of the shell-and-tube reactor) generally made of steel. The wall thickness of the reaction tubes is typically from 1 to 3 mm. Their diameter is generally (uniformly) from 10 to 50 mm or from 15 to 40 mm, frequently from 20 to 30 mm. The number of reaction tubes accommodated in the shell-and-tube reactor is generally at least 1000, or 3000, or 5000, preferably at least 10 000. The number of reaction tubes accommodated in the shell-and-tube reactor is frequently from 15 000 to 30 000 or up to 40 000 or up to 50 000. The length of the reaction tubes is normally up to a few meters; a typical reaction tube length is in the range from 1 to 8 m, frequently from 2 to 7 m, often from 2.5 to 6m.

Furthermore, the catalyst bed installed in the ODH-reactor 1 can be made up of a single zone or of 2 or more zones. These zones can consist of pure catalyst or be diluted with a material which does not react with the starting gas or components of the product gas of the reaction. Furthermore, the catalyst beds can consist of all-active catalysts or supported coated catalysts.

The product gas stream 2 leaving the oxidative dehydrogenation generally comprises butadiene together with unreacted 1-butene and 2-butene, oxygen and water vapor. Furthermore, it generally comprises, as secondary component, carbon monoxide, carbon dioxide, inert gases (mainly nitrogen), low-boiling hydrocarbons such as methane, ethane, ethene, propane and propene, butane and isobutane, possibly hydrogen and possibly oxygen-comprising hydrocarbons, known as oxygenates. Oxygenates can be, for example, formaldehyde, furan, acetic acid, maleic anhydride, formic acid, methacrolein, methacrylic acid, crotonaldehyde, crotonic acid, propionic acid, acrylic acid, methyl vinyl ketone, styrene, benzaldehyde, benzoic acid, phthalic anhydride, fluorenone, anthraquinone and butyraldehyde.

The product gas stream 2 at the reactor outlet has a temperature close to the temperature at the end of the catalyst bed. The product gas stream is then brought to a temperature of 150-400° C., preferably 160-300° C., particularly preferably 170-250° C. It is possible to insulate the line through which the product gas stream flows or use a heat exchanger in order to maintain the temperature in the desired range. This heat exchanger system can be of any desired design as long as the system enables the temperature of the product gas to be kept at the desired level. Examples of heat exchangers are coil heat exchangers, plate heat exchangers, double-tube heat exchangers, multitube heat exchangers, boiler coil heat exchangers, boiler wall heat exchangers, liquid-liquid contact heat exchangers, air heat exchangers, direct contact heat exchangers and finned tube heat exchangers. Since, while the temperature of the product gas is set to the desired temperature, part of the high-boiling by-products comprised in the product gas can condense out, the heat exchanger system should preferably have two or more heat exchangers. If two or more of the heat exchangers provided are arranged in parallel and distributed cooling of the product gas obtained is made possible in the heat exchangers, the amount of high-boiling by-products which precipitate in the heat exchangers decreases and the operating time of the heat exchangers can thus be increased. As an alternative to the abovementioned method, the two or more heat exchangers provided can be arranged in parallel. The product gas is introduced into one or more but not all heat exchangers which are after a particular time of operation relieved by other heat exchangers. This method enables cooling to be continued, part of the heat of the reaction to be recovered and, in parallel thereto, the high-boiling by-products precipitated in one of the heat exchangers can be removed. As an organic solvent as mentioned above, it is possible to use a solvent which is able to dissolve the high-boiling by-products. Examples are aromatic hydrocarbon solvents such as toluene and xylenes and also alkaline aqueous solvents such as an aqueous solution of sodium hydroxide.

A major part of the high-boiling secondary components and of the water is subsequently separated off from the product gas stream 2 by cooling and compression. Cooling is effected, according to the invention, by bringing into contact with an organic solvent. This step will hereinafter also be referred to as a quench. This quench can consist of only one stage (3 in FIG. 1) or of a plurality of stages (for example 3, 7 in FIG. 1). The product gas stream 2 is thus brought into direct contact with an organic solvent as cooling medium 4 and cooled thereby. Cooling media used according to the invention are organic solvents, preferably aromatic hydrocarbons, particularly preferably toluene, o-xylene, m-xylene, p-xylene or mixtures thereof.

Preference is given to a two-stage quench (comprising the stages 3 and 7 as shown in FIG. 1), i.e. step Ca) comprises two cooling stages Ca1) and Ca2) in which the product gas stream b is brought into contact with the organic solvent.

In general, the product gas 2 has, depending on the presence and temperature level of a heat exchanger before the quench 3, a temperature of 100-440° C. In the 1st quenching stage 3, the product gas is brought into contact with the cooling medium composed of an organic solvent. Here, the cooling medium can be introduced via a nozzle in order to achieve very efficient mixing with the product gas. For the same purpose, internals such as further nozzles through which the product gas and the cooling medium pass together can be introduced into the quenching stage. The coolant inlet into the quench is designed so that blockages due to deposits in the region of the coolant inlet are minimized.

In general, the product gas 2 is cooled to 5-180° C., preferably to 30-130° C. and even more preferably to 60-110° C., in the first quenching stage 3. The temperature of the cooling medium 4 at the inlet can generally be 25-200° C., preferably 40-120° C., in particular 50-90° C. The pressure in the first quenching stage 3 is not subject to any particular restrictions, but is generally 0.01-4 bar (gauge), preferably 0.1-2 bar (gauge) and particularly preferably 0.2-1 bar (gauge). When relatively large amounts of high-boiling by-products are present in the product gas, polymerization of high-boiling by-products and deposition of solids caused by high-boiling by-products in this process section can easily occur. In general, quenching stage 3 is configured as a cooling tower. The cooling medium 4 used in the cooling tower is frequently circulated in a quench circuit. The circulating flow of the cooling medium in liters per hour, based on the mass flow of butadiene in grams per hour, can generally be 0.0001-5 l/g, preferably 0.001-1 l/g and particularly preferably 0.002-0.2 l/g.

The temperature of the cooling medium 4 at the bottom can generally be 27-210° C., preferably 45-130° C., in particular 55-95° C. Since the loading of the cooling medium 4 with secondary components increases over the course of time, part of the loaded cooling medium can be taken off from the circuit as purge stream 4a and the circulating amount can be kept constant by addition of unloaded cooling medium 4b. The ratio of amount discharged and amount added depends on the vapor loading of the product gas and on the product gas temperature at the end of the first quenching stage.

Depending on the temperature, pressure and water content of the product gas 2, condensation of water can occur in the first quenching stage 3. In this case, an additional aqueous phase 5 additionally comprising water-soluble secondary components can be formed. This can then be taken off at the bottom of the quenching stage 3. The aqueous phase can also be separated off in an additional phase separator. This can, for example, be located within the quench circuit. The aqueous phase can be taken off or at least partly recirculated to the quench. The cooling medium 4 can also be taken off or at least partly recirculated to the quench. As an alternative, the phase separator can, for example, be located in the purge stream 4a. The aqueous phase can be taken off or at least partly recirculated to the quench. The cooling medium 4 can be at least partly recirculated to the quench. Depending on mixing of the aqueous and organic phases in the quench, in particular in the bottoms, the proportion of aqueous phase in the recirculation of the quench circuit can in this case be several hundred ppm by volume, possibly several % by volume or the quench circuit can consist largely of the aqueous phase.

Preference is given to a mode of operation in which no aqueous phase is formed in the first quenching stage 3.

The cooled product gas stream 6, which is possibly depleted in secondary components, can then be fed to a second quenching stage 7. In this, it can be brought into contact again with a cooling medium 8.

As cooling medium 8, use is made according to the invention of organic solvents, preferably aromatic hydrocarbon solvents, particularly preferably toluene, o-xylene, m-xylene, p-xylene and mixtures thereof.

In general, the product gas is cooled to from 5 to 100° C., preferably to 15-85° C. and even more preferably to 30-70° C., up to the gas outlet from the second quenching stage 7. The coolant can be introduced in countercurrent to the product gas. In this case, the temperature of the cooling medium 8 at the coolant inlet can be 5-100° C., preferably 15-85° C., in particular 30-70° C. The pressure in the second quenching stage 7 is not subject to any particular restrictions, but is generally 0.01-4 bar (gauge), preferably 0.1-2 bar (gauge) and particularly preferably 0.2-1 bar (gauge). The second quenching stage 7 is preferably configured as a cooling tower. The cooling medium 8 used in the cooling tower is frequently circulated in a quench circuit. The circulating flow of the cooling medium 8 in liters per hour, based on the mass flow of butadiene in grams per hour, can generally be 0.0001-51/g, preferably 0.001-1 l/g and particularly preferably 0.002-0.21/g.

Depending on temperature, pressure and water content the product gas 6, condensation of water can occur in the second quenching stage 7. In this case, an additional aqueous phase 9 which can additionally comprise water-soluble secondary components can be formed. This can then be taken off at the bottom of the quenching stage 7. The aqueous phase can also be separated off in an additional phase separator. This can, for example, be located within the quench circuit. The aqueous phase can be taken off or at least partly recirculated to the quench. The cooling medium 8 can also be at least partly recirculated to the quench. In this case, the proportion of aqueous phase in the recirculation of the quench circuit is low.

As an alternative, the phase separator can, for example, be located in the purge stream 8a. The aqueous phase can be taken off or at least partly recirculated to the quench. The cooling medium 8 can be at least partly recirculated to the quench. Depending on mixing of the aqueous and organic phases in the quench, in particular in the bottoms, the proportion of aqueous phase in the recirculation of the quench circuit can in this case be several hundred ppm by volume, possibly several % by volume or the quench circuit can consist largely of the aqueous phase.

The temperature of the cooling medium 8 at the bottom can generally be 20-210° C., preferably 35-120° C., in particular 45-85° C. Since the loading of the cooling medium 8 with secondary components increases over the course of time, part of the loaded cooling medium can be taken off as purge stream 8a from the circuit and the circulating amount can be kept constant by addition of unloaded cooling medium 8b.

To achieve very good contact between product gas and cooling medium, internals can be present in the second quenching stage 8. Such internals comprise, for example, bubble cap trays, centrifugal trays and/or sieve trays, columns having structured packings, e.g. sheet metal packings having a specific surface area of from 100 to 1000 m²/m³, e.g. Mellapak® 250 Y, and columns packed with random packing elements.

The solvent circuits of the two quenching stages can either be separate from one another or connected to one another. Thus, for example, stream 8a can be added to stream 4b or replace the latter. The desired temperature of the circulating streams can be set by means of suitable heat exchangers.

In a preferred embodiment of the invention, the cooling step Ca) is thus carried out in two stages, with the solvent laden with secondary components from the second stage Ca2) being introduced into the first stage Ca1). The solvent taken off from the second stage Ca2) comprises a smaller amount of secondary components than the solvent taken off from the first stage Ca1).

To minimize entrainment of liquid constituents from the quench into the offgas line, suitable constructional measures, for example installation of a demister, can be carried out. Furthermore, high-boiling substances which are not separated off from the product gas in the quench can be removed from the product gas by means of further constructional measures, for example further gas scrubs.

A gas stream 10 comprising n-butane, 1-butene, 2-butenes, butadiene, possibly oxygen, hydrogen, water vapor, small amounts of methane, ethane, ethene, propane and propene, isobutane, carbon oxides, inert gases and parts of the solvent used in the quench is obtained. Furthermore, traces of high-boiling components which are not quantitatively separated off in the quench can remain in this gas stream 10.

The gas stream b from the cooling step Ca), which is depleted in high-boiling secondary components, is subsequently cooled in step (b) in at least one compression stage Cba) and preferably in at least one cooling stage Cbb) by contacting with an organic solvent as coolant.

The product gas stream 10 from the solvent quench (3 or preferably 3 and 7) is compressed in at least one compression stage 11 and subsequently cooled further in the cooling apparatus 13, forming at least one condensate stream 15 comprising water. Furthermore, the solvent used in the solvent quench condenses out and forms a separate phase 14. This leaves a gas stream 16 comprising butadiene, 1-butene, 2-butenes, oxygen, water vapor, possibly low-boiling hydrocarbons such as methane, ethane, ethene, propane and propene, butane and isobutane, possibly carbon oxides and possibly inert gases. Furthermore, this product gas stream can further comprise traces of high-boiling components.

The compression and cooling of the gas stream 10 can be carried out in one or more stages (n-stage). In general, the stream is compressed overall from a pressure in the range from 1.0 to 4.0 bar (absolute) to a pressure in the range from 3.5 to 20 bar (absolute). Each compression stage is followed by a cooling stage in which the gas stream is cooled to a temperature in the range from 15 to 60° C. The condensate stream can thus also comprise a plurality of streams in the case of multistage compression. The condensate stream comprises largely water (aqueous phase 15) and the solvent used in the quench (organic phase 14). The two streams (aqueous and organic phases) can additionally comprise small amounts of secondary components such as low boilers, C₄-hydrocarbons, oxygenates and carbon oxides.

To cool the stream 12 and/or to remove further secondary components from the stream 12, the condensed quench solvent 14 can be cooled in a heat exchanger and recirculated as coolant to the apparatus 13. Since the loading of this cooling medium 14 with secondary components increases over the course of time, part of the loaded cooling medium (14a) can be taken off from the circuit and the circulating amount of cooling medium can be kept constant by addition of unloaded solvent (14b).

The solvent 14b which is added as cooling medium thus likewise preferably consists of the aromatic hydrocarbon solvent used as quench solvent.

The condensate stream 14a can be recirculated into the circulating stream 4b and/or 8b of the quench. In this way, the C4 component absorbed in the condensate stream 14a can be brought back into the gas stream and the yield can thus be increased. Suitable compressors are, for example, turbocompressors, rotary piston compressors and reciprocating piston compressors. The compressors can be driven, for example, by means of an electric motor, expander or a gas or steam turbine. Typical compression ratios (exit pressure:entry pressure) per compression stage are, depending on the construction type, in the range from 1.5 to 3.0. Cooling of the compressed gas is effected using heat exchangers flushed with an organic solvent or organic quenching stages which can be configured, for example, as shell-and-tube heat exchangers, coil heat exchangers or plate heat exchangers. Coolants used in the heat exchangers are cooling water or heat transfer oils. In addition, preference is given to air cooling using blowers.

The gas stream 16 comprising butadiene, n-butenes, oxygen, low-boiling hydrocarbons (methane, ethane, ethene, propane, propene, n-butane, isobutane), possibly water vapor, possibly carbon oxides and possibly inert gases and possibly traces of secondary components is fed as starting stream to the further work-up.

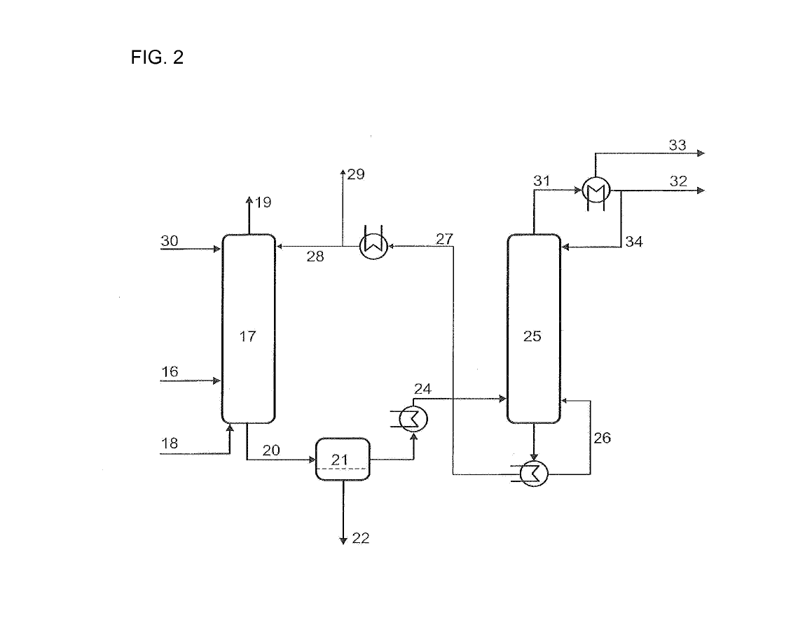

In a step D), incondensable and low-boiling gas constituents comprising oxygen, low-boiling hydrocarbons (methane, ethane, ethene, propane, propene), carbon oxides and inert gases are separated off as gas stream 19 from the process gas stream 16 by absorption of the C₄-hydrocarbons in a high-boiling absorption medium (28 and/or 30) in an absorption column 17 and subsequent desorption of the C₄-hydrocarbons. Step D) preferably comprises, as shown in FIG. 2, the steps Da) to Dc):
Da) absorption of the C₄-hydrocarbons comprising butadiene and n-butenes in a high-boiling absorption medium (28 and/or 30), giving an absorption medium stream loaded with C₄-hydrocarbons and the gas stream 19,
Db) removal of oxygen from the absorption medium stream loaded with C₄-hydrocarbons from step Da) by stripping with an incondensable gas stream 18, giving an absorption medium stream 20 loaded with C₄-hydrocarbons, and
Dc) desorption of the C₄-hydrocarbons from the loaded absorption medium stream, giving a C₄ product gas stream 31 consisting essentially of C₄-hydrocarbons.

For this purpose, the gas stream 16 is brought into contact with an inert absorption medium in the absorption step 17 and the C₄-hydrocarbons are absorbed in the inert absorption medium, giving an absorption medium loaded with C₄-hydrocarbons and an off gas 19 comprising the remaining gas constituents. In a desorption stage, the C₄-hydrocarbons are liberated again from the high-boiling absorption medium.

The absorption stage can be carried out in any suitable absorption column known to those skilled in the art. The absorption can be carried out by simply passing the product gas stream through the absorption medium. However, it can also be carried out in columns or in rotational absorbers. It can be carried out in cocurrent, countercurrent or cross-current. The absorption is preferably carried out in countercurrent. Suitable absorption columns are, for example, tray columns having bubble cap trays, centrifugal trays and/or sieve trays, columns having structured packings, e.g. sheet metal packings having a specific surface area of from 100 to 1000 m²/m³, e.g. Mellapak® 250 Y, and columns packed with random packing elements. However, trickle towers and spray towers, graphite block absorbers, surface absorbers such as thick film and thin film absorbers and also rotary columns, plate scrubbers, cross-spray scrubbers and rotational scrubbers are also possible.

In an embodiment, the gas stream 16 comprising butadiene, n-butenes and the low-boiling and incondensable gas constituents is fed into the lower region of an absorption column. In the upper region of the absorption column, the high-boiling absorption medium (28 and/or 30) is introduced.

Inert absorption media used in the absorption stage are generally high-boiling nonpolar solvents in which the C₄-hydrocarbon mixture to be separated off has a significantly greater solubility than do the remaining gas constituents to be separated off. Suitable absorption media are comparatively nonpolar organic solvents, for example aliphatic C₈-C₁₈-alkanes or aromatic hydrocarbons such as middle oil fractions from paraffin distillation, toluene or ethers having bulky groups, or mixtures of these solvents, with a polar solvent such as 1,2-dimethyl phthalate being able to be added to these. Further suitable absorption media are esters of benzoic acid and phthalic acid with straight-chain C₁-C₈-alkanols and also heat transfer oils such as biphenyl and diphenyl ether, chloro derivatives thereof and also triarylalkenes. One suitable absorption medium is a mixture of biphenyl and diphenyl ether, preferably having the azeotropic composition, for example the commercially available Diphyl®. This solvent mixture frequently comprises dimethyl phthalate in an amount of from 0.1 to 25% by weight.

In a preferred embodiment, the same solvent is used in the absorption stage Da) as in the cooling stage Ca).

Preferred absorption media are solvents which have a solvent capability for organic peroxides of at least 1000 ppm (mg of active oxygen/kg of solvent). In the preferred embodiment, toluene is used as solvent for the absorption.

At the top of the absorption column 17, an offgas stream 19 which comprises essentially oxygen, low-boiling hydrocarbons (methane, ethane, ethene, propane, propene), possibly C₄-hydrocarbons (butane, butenes, butadiene), possibly inert gases, possibly carbon oxides and possibly also water vapor is taken off. Part of this stream can be fed to the ODH reactor. This allows, for example, the stream entering the ODH reactor to be set to the desired C₄-hydrocarbon content.

At the bottom of the absorption column, residues of oxygen dissolved in the absorption medium are discharged by flushing with a gas 18 in a further column. The remaining proportion of oxygen should be so small that the stream 31 comprising butane, butene and butadiene leaving the desorption column comprises a maximum of 100 ppm of oxygen.

Stripping-out of the oxygen in step Db) can be carried out in any suitable column known to those skilled in the art. Stripping can be effected by simply passing incondensable gases, preferably inert gases such as nitrogen, through the loaded absorption solution. C₄-hydrocarbons which are also stripped out are scrubbed back into the absorption solution in the upper part of the absorption column 17 by recirculating the gas stream back into this absorption column. This can be effected both by provision of tubes in the stripper column or by direct mounting of the stripper column below the absorber column. Since the pressure in the stripping column section and the absorption column section is the same according to the invention, these can be directly coupled. Suitable stripping columns are, for example, tray columns having bubble cap trays, centrifugal trays and/or sieve trays, columns having structured packings, e.g. sheet metal packings having a specific surface area of from 100 to 1000 m²/m³, e.g. Mellapak® 250 Y, and columns packed with random packing elements. However, trickle towers and spray towers and also rotary columns, plate scrubbers, cross-spray scrubbers and rotational scrubbers are also possible. Suitable gases are, for example, nitrogen or methane.

The absorption medium stream 20 loaded with C₄-hydrocarbons comprises water. This is separated off as stream 22 from the absorption medium in a decanter 21 so as to give a stream 23 which comprises only the water dissolved in the absorption medium.

The absorption medium stream 23 which is loaded with C₄-hydrocarbons and has been very largely freed of water can be heated in a heat exchanger and subsequently introduced as stream 24 into a desorption column 25. In one process variant, the desorption step Dc) is carried out by depressurization and/or heating of the loaded absorption medium. A preferred process variant is utilization of a reboiler in the bottom of the desorption column 25.

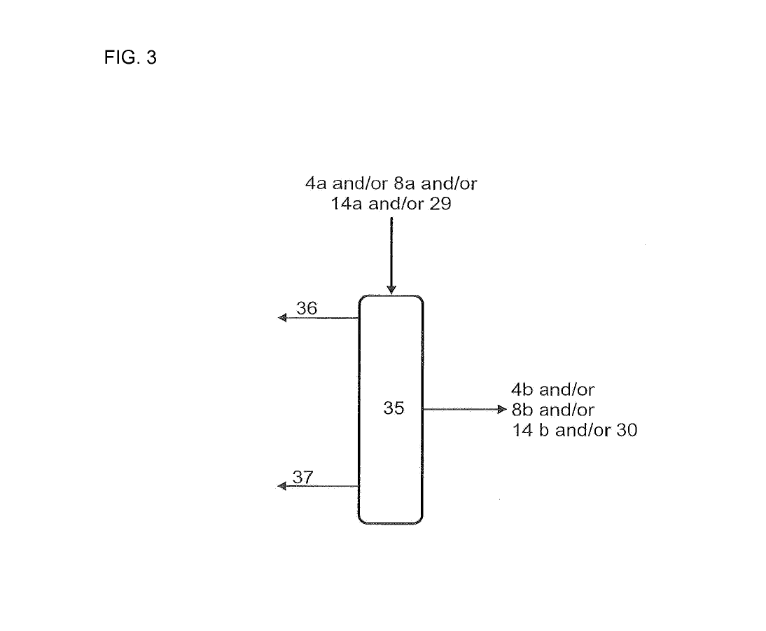

The absorption medium 27 which has been regenerated in the desorption stage can be cooled in a heat exchanger and recirculated as stream 28 to the absorption stage 17. Low boilers such as ethane or propane and also high-boiling components such as benzaldehyde, maleic anhydride and phthalic anhydride which are present in the process gas stream can accumulate in the recycle stream. In order to limit accumulation, a purge stream 29 can be taken off. This can be separated either alone or combined with streams 14a and/or 8b and/or 4b in a distillation column 35 (FIG. 3) according to the prior art into low boilers 36, regenerated absorbent 30 and/or regenerated cooling medium 4b and/or 8b and/or 14b (FIGS. 2 and 3) and high boilers 37. It can preferably be introduced directly into streams 14b and/or 8b and/or 4b in order to scrub the C₄-hydrocarbons still dissolved in the stream 29 back into the process gas stream. When stream 29 is separated in the distillation column 35, the streams 36 and 37 can, for example, be incinerated and thus utilized to generate energy. Of course, the streams 14a and/or 8a and/or 4a can also be purified without stream 29 in a distillation column 35 and be recirculated to the process.

The C₄ product gas stream 31 consisting essentially of n-butane, n-butenes and butadiene generally comprises from 20 to 80% by volume of butadiene, from 0 to 80% by volume of n-butane, from 0 to 10% by volume of 1-butene and from 0 to 50% by volume of 2-butenes, where the total amount is 100% by volume. Furthermore, small amounts of isobutane can be comprised.

Part of the condensed overhead output comprising mainly C₄-hydrocarbons from the desorption column is recirculated as stream 34 to the top of the column in order to increase the separation performance of the column.

The liquid (stream 32) or gaseous (stream 33) C₄ product streams leaving the condenser are subsequently separated by extractive distillation in step E) using a solvent selective for butadiene into a stream comprising butadiene and the selective solvent and a stream comprising n-butenes.

The extractive distillation can, for example, be carried out as described in “Erdöl and Kohle-Erdgas-Petrochemie”, volume 34 (8), pages 343 to 346 or “Ullmanns Enzyklopädie der Technischen Chemie”, volume 9, 4^{th} edition 1975, pages 1 to 18. For this purpose, the C₄ product gas stream is brought into contact with an extractant, preferably an N-methylpyrrolidone (NMP)/water mixture, in an extraction zone. The extraction zone is generally configured in the form of a scrubbing column comprising trays, random packing elements or ordered packing as internals. This generally has from 30 to 70 theoretical plates so as to achieve a sufficiently good separation performance. The scrubbing column preferably has a backwashing zone at the top of the column. This backwashing zone serves to recover the extractant comprised in the gas phase by means of a liquid hydrocarbon runback, for which purpose the overhead fraction is condensed beforehand. The mass ratio of extractant to C₄ product gas stream in the feed to the extraction zone is generally from 10:1 to 20:1. The extractive distillation is preferably carried out at a temperature at the bottom in the range from 100 to 250° C., in particular at a temperature in the range from 110 to 210° C., a temperature at the top in the range from 10 to 100° C., in particular in the range from 20 to 70° C., and a pressure in the range from 1 to 15 bar, in particular in the range from 3 to 8 bar. The extractive distillation column preferably has from 5 to 70 theoretical plates.

Suitable extractants are butyrolactone, nitriles such as acetonitrile, propionitrile, methoxypropionitrile, ketones such as acetone, furfural, N-alkyl-substituted lower aliphatic acid amides such as dimethylformamide, diethylformamide, dimethylacetamide, diethylacetamide, N-formylmorpholine, N-alkyl-substituted cyclic acid amides (lactams) such as N-alkylpyrrolidones, in particular N-methylpyrrolidone (NMP). In general, use is made of alkyl-substituted lower aliphatic acid amides or N-alkyl-substituted cyclic acid amides. Dimethylformamide, acetonitrile, furfural and in particular NMP are particularly advantageous.

It is also possible to use mixtures of these extractants with one another, e.g. NMP and acetonitrile, mixtures of these extractants with cosolvents and/or tert-butyl ethers, e.g. methyl tert-butyl ether, ethyl tert-butyl ether, propyl tert-butyl ether, n-butyl or isobutyl tert-butyl ether. NMP is particularly useful, preferably in aqueous solution, preferably with from 0 to 20% by weight of water, particularly preferably with from 7 to 10% by weight of water, in particular with 8.3% by weight of water.

The overhead product stream from the extractive distillation column comprises essentially butane and butenes and small amounts of butadiene and is taken off in gaseous or liquid form. In general, the stream consisting essentially of n-butane and 2-butene comprises up to 100% by volume of n-butane, from 0 to 50% by volume of 2-butene and from 0 to 3% by volume of further constituents such as isobutane, isobutene, propane, propene and C₅⁺-hydrocarbons.

The stream consisting essentially of n-butane and 2-butene can be added in its entirety or in part to the C₄ feed to the ODH reactor. Since the butene isomers of this recycle stream consist essentially of 2-butenes and 2-butenes are generally oxidatively dehydrogenated to butadiene more slowly than 1-butene, this recycle stream can be catalytically isomerized before introduction into the ODH reactor. In this way, the isomer distribution can be set to the isomer distribution present at thermodynamic equilibrium.

In a step F), the stream comprising butadiene and the selective solvent is separated by distillation into a stream consisting essentially of the selective solvent and a butadiene-comprising stream.

The stream obtained at the bottom of the extractive distillation column generally comprises the extractant, water, butadiene and small proportions of butenes and butane and is fed to a distillation column. In this, butadiene can be obtained at the top or as a side offtake stream. A stream comprising extractant and possibly water is obtained at the bottom of the distillation column, with the composition of the stream comprising extractant and water corresponding to the composition introduced into the extraction. The stream comprising extractant and water is preferably recirculated to the extractive distillation.

If butadiene is isolated via a side offtake, the extraction solution taken off in this way is transferred to a desorption zone in which the butadiene is desorbed again from the extraction solution and backwashed. The desorption zone can, for example, be in the form of a scrubbing column which has from 2 to 30, preferably from 5 to 20, theoretical plates and optionally a backwashing zone having, for example, 4 theoretical plates. This backwashing zone serves to recover the extractant comprised in the gas phase by means of a liquid hyrocarbon runback, for which purpose the overhead fraction is condensed beforehand. Ordered packings, trays or random packing elements are provided as internals. The distillation is preferably carried out at a temperature at the bottom in the range from 100 to 300° C., in particular in the rang from 150 to 200° C., and a temperature at the top in the range from 0 to 70° C., in particular in the range from 10 to 50° C. The pressure in the distillation column is preferably in the range from 1 to 10 bar. In general, a reduced pressure and/or an elevated temperature compared to the extraction zone prevail in the desorption zone.

The desired product stream obtained at the top of the column generally comprises from 90 to 100% by volume of butadiene, from 0 to 10% by volume of 2-butene and from 0 to 10% by volume of n-butane and isobutane. To purify the butadiene further, it is possible to carry out a further distillation according to the prior art.

### EXAMPLE

The example describes the use of toluene phases both in the quenching and compression stages and in the C₄ absorption. In this way excellent dissolution of a number of high-boiling secondary components is effected in the quench and deposits downstream of the quench are prevented. In addition, the loss of C₄-hydrocarbons dissolved in the discharged toluene is minimized by recirculation of the purge streams from the second quenching part, the intermediate cooler of the compressor and the C₄-absorption/desorption in the recycle streams further upstream in the process.

A process gas 2 with a temperature of 210° C., a pressure of 1.3 bar and the composition shown in Table 1 is provided from the ODH reactor 1. This gas stream is brought to a temperature of 60° C. in the quenching section 3 by means of a toluene recycle stream having a temperature of 35° C. and a composition as shown in Table 1 in stream 4. Here, a number of the secondary components are dissolved out of the gas stream and the composition of the process gas stream changes to the concentrations shown for stream 6. The mass ratio of the recycle stream 4 to process gas 2 and to the purge stream 4a is 1:0.2:0.0033. Stream 4b comprises the purge streams 8a (2nd quenching stage) and a make-up stream composed of fresh toluene.

The gas stream 6 is cooled further to 40° C. in the second quenching stage 7 by means of a further toluene recycle stream 8 which enters the quench at the top end with a temperature of 35° C. The resulting gas stream 10 has the composition shown in Table 1, while the mass ratio of stream 6 to 8 is 1:5.6 and a purge stream 8a having a proportion of 2% of stream 8 is taken off and introduced into the first quenching section. The stream 8b comprises 0.2% of the purge stream 29 from the C4 absorption and 51.6% of the streams 14a, 14a′, 14a″ and 14a′″ (purge stream from the heat exchanger downstream of the compressor stages 1 to 4) and 48.2% of fresh toluene.

The gas stream 10 is compressed to 10 bar absolute in a 4-stage compressor with the intermediate coolers shown in FIG. 1. The purge streams 14a, 14a′, 14a″ and 14a′″ are all added to the stream 8b.

The resulting gas stream 16′″ (outlet stream from the heat exchanger 13′″ downstream of the 4^{th} compression stage) has a temperature of 35° C. and the composition shown in Table 1. This stream is separated in the absorber column 17 by means of the absorption medium stream 28 conveyed in countercurrent and entering the column at 10 bar absolute and 35° C. at the top of the column into a gas stream 19 and an absorption medium stream loaded mainly with C4-hydrocarbons. The loaded absorption medium stream is freed of oxygen by means of a stream 18 comprising nitrogen and having a temperature of 35° C. to such an extent that the gas stream 33 leaving the desorber column has an oxygen content of only 10 ppm. The mass ratio of stream 16′″ to stream 28 is 1:2.48 and the mass ratio of 28 to 18 is 1:0.006. A stream 22 comprising mainly water is taken off from stream 20; this represents a proportion of 0.002% of stream 20 and its precise composition is shown in Table 2.

The resulting stream 23 is heated to 120° C. and introduced as stream 24 into the desorber column 25 at a pressure at the top of 5.5 bar absolute. The mass flow ratio of stream 26 having a temperature of 175° C. to stream 27 is 1:100.

The mass flow ratio of overhead streams 31 and 34 is 1:0.56 and the product stream 33 has the composition shown in Table 2 and is passed on to the above-described extractive distillation. The stream 32 does not occur in this example.

TABLE 1

| | | | | | 4b | | | | | |
|---|---|---|---|---|---|---|---|---|---|---|
| | | | | | o. | | | | | |
| | 2 | 4a | 4 | 5 | 8a | 6 | 8 | 9 | 10 | 16′″ |
| Butane | 3.44 | 0.21 | 0.21 | 0.00 | 0.30 | 2.43 | 0.32 | 0.00 | 3.42 | 3.84 |
| l-Butane | 0.41 | 0.02 | 0.02 | 0.00 | 0.02 | 0.28 | 0.02 | 0.00 | 0.40 | 0.45 |
| l-Butene | 0.02 | 0.00 | 0.00 | 0.00 | 0.00 | 0.02 | 0.00 | 0.00 | 0.02 | 0.03 |
| 1-Butene | 0.02 | 0.00 | 0.00 | 0.00 | 0.00 | 0.01 | 0.00 | 0.00 | 0.02 | 0.02 |
| C-2-Butene | 0.41 | 0.03 | 0.03 | 0.00 | 0.04 | 0.29 | 0.04 | 0.00 | 0.41 | 0.45 |
| T-2-Butene | 1.06 | 0.07 | 0.07 | 0.00 | 0.10 | 0.75 | 0.10 | 0.00 | 1.06 | 1.18 |
| 1,3-Butadiene | 11.34 | 0.59 | 0.59 | 0.00 | 0.84 | 7.93 | 0.89 | 0.00 | 11.18 | 12.67 |
| Water | 12.16 | 0.13 | 0.13 | 96.79 | 0.11 | 7.21 | 0.43 | 99.53 | 2.76 | 0.32 |
| Ethene | 0.00 | 0.00 | 0.00 | 0.00 | 0.00 | 0.00 | 0.00 | 0.00 | 0.00 | 0.00 |
| 4-Vinylcyclo-hexene | 0.00 | 0.01 | 0.01 | 0.00 | 0.00 | 0.00 | 0.00 | 0.00 | 0.00 | 0.00 |
| Toluene | 0.00 | 97.58 | 97.59 | 0.11 | 98.39 | 33.37 | 98.00 | 0.10 | 14.51 | 1.24 |
| Maleic anhydride | 0.12 | 0.49 | 0.48 | 2.30 | 0.00 | 0.00 | 0.00 | 0.02 | 0.00 | 0.00 |
| Benzoic acid | 0.04 | 0.23 | 0.23 | 0.00 | 0.00 | 0.00 | 0.00 | 0.00 | 0.00 | 0.00 |
| Benzaldehyde | 0.06 | 0.37 | 0.36 | 0.00 | 0.02 | 0.01 | 0.02 | 0.00 | 0.00 | 0.00 |
| Acetic acid | 0.02 | 0.04 | 0.04 | 0.37 | 0.01 | 0.01 | 0.01 | 0.12 | 0.00 | 0.00 |
| Anthraquinone | 0.00 | 0.03 | 0.03 | 0.00 | 0.00 | 0.00 | 0.00 | 0.00 | 0.00 | 0.00 |
| Styrene | 0.01 | 0.05 | 0.05 | 0.00 | 0.01 | 0.01 | 0.01 | 0.00 | 0.00 | 0.00 |
| Ethylbenzene | 0.00 | 0.01 | 0.01 | 0.00 | 0.00 | 0.00 | 0.00 | 0.00 | 0.00 | 0.00 |
| Crotonic acid | 0.00 | 0.01 | 0.01 | 0.00 | 0.00 | 0.00 | 0.00 | 0.00 | 0.00 | 0.00 |
| Propionic acid | 0.03 | 0.08 | 0.08 | 0.31 | 0.02 | 0.02 | 0.02 | 0.10 | 0.00 | 0.00 |
| Furan | 0.22 | 0.04 | 0.04 | 0.00 | 0.08 | 0.18 | 0.08 | 0.00 | 0.25 | 0.24 |
| CO₂ | 2.48 | 0.01 | 0.01 | 0.03 | 0.01 | 1.67 | 0.01 | 0.04 | 2.32 | 2.78 |
| N₂ | 61.54 | 0.01 | 0.01 | 0.07 | 0.02 | 41.36 | 0.02 | 0.07 | 57.49 | 69.32 |
| O₂ | 6.61 | 0.00 | 0.00 | 0.01 | 0.00 | 4.44 | 0.00 | 0.01 | 6.18 | 7.45 |

TABLE 2

| | 19 | 20 | 22 | 24 | 26 | 27 | 28 | 29 | 31 | 33 | 34 |
|---|---|---|---|---|---|---|---|---|---|---|---|
| Butane | 0.21 | 1.56 | 0.01 | 1.56 | 2.06 | 0.20 | 0.20 | 0.20 | 19.54 | 19.52 | 19.56 |
| l-Butane | 0.03 | 0.18 | 0.00 | 0.18 | 0.23 | 0.02 | 0.02 | 0.02 | 1.93 | 2.30 | 1.63 |
| l-Butene | 0.00 | 0.01 | 0.00 | 0.01 | 0.01 | 0.00 | 0.00 | 0.00 | 0.12 | 0.13 | 0.12 |
| 1-Butene | 0.00 | 0.01 | 0.00 | 0.01 | 0.01 | 0.00 | 0.00 | 0.00 | 0.10 | 0.11 | 0.10 |
| C-2-Butene | 0.02 | 0.19 | 0.01 | 0.19 | 0.24 | 0.02 | 0.02 | 0.02 | 2.60 | 2.31 | 2.83 |
| T-2-Butene | 0.06 | 0.48 | 0.02 | 0.48 | 0.64 | 0.06 | 0.06 | 0.06 | 6.51 | 6.03 | 6.90 |
| 1,3-Butadiene | 0.69 | 5.07 | 0.01 | 5.07 | 6.64 | 0.57 | 0.57 | 0.57 | 62.02 | 64.41 | 60.12 |
| Water | 0.22 | 0.05 | 98.83 | 0.05 | 0.06 | 0.00 | 0.00 | 0.00 | 0.35 | 0.66 | 0.09 |
| Ethene | 0.00 | 0.00 | 0.00 | 0.00 | 0.00 | 0.00 | 0.00 | 0.00 | 0.00 | 0.00 | 0.00 |
| 4-Vinylcyclo-hexene | 0.00 | 0.00 | 0.00 | 0.00 | 0.00 | 0.00 | 0.00 | 0.00 | 0.00 | 0.00 | 0.00 |
| Toluene | 1.97 | 92.12 | 0.06 | 92.12 | 89.86 | 99.10 | 99.10 | 99.10 | 2.81 | 0.11 | 4.95 |
| Maleic anhydride | 0.00 | 0.00 | 0.00 | 0.00 | 0.00 | 0.00 | 0.00 | 0.00 | 0.00 | 0.00 | 0.00 |
| Benzoic acid | 0.00 | 0.00 | 0.00 | 0.00 | 0.00 | 0.00 | 0.00 | 0.00 | 0.00 | 0.00 | 0.00 |
| Benzaldehyde | 0.00 | 0.00 | 0.00 | 0.00 | 0.00 | 0.00 | 0.00 | 0.00 | 0.00 | 0.00 | 0.00 |
| Acetic acid | 0.00 | 0.00 | 0.00 | 0.00 | 0.00 | 0.00 | 0.00 | 0.00 | 0.00 | 0.00 | 0.00 |
| Anthraquinone | 0.00 | 0.00 | 0.00 | 0.00 | 0.00 | 0.00 | 0.00 | 0.00 | 0.00 | 0.00 | 0.00 |
| Styrene | 0.00 | 0.00 | 0.00 | 0.00 | 0.00 | 0.00 | 0.00 | 0.00 | 0.00 | 0.00 | 0.00 |
| Ethylbenzene | 0.00 | 0.00 | 0.00 | 0.00 | 0.00 | 0.00 | 0.00 | 0.00 | 0.00 | 0.00 | 0.00 |
| Crotonic acid | 0.00 | 0.00 | 0.00 | 0.00 | 0.00 | 0.00 | 0.00 | 0.00 | 0.00 | 0.00 | 0.00 |
| Propionic acid | 0.00 | 0.00 | 0.00 | 0.00 | 0.00 | 0.00 | 0.00 | 0.00 | 0.00 | 0.00 | 0.01 |
| Furan | 0.01 | 0.11 | 0.00 | 0.11 | 0.15 | 0.03 | 0.03 | 0.03 | 2.59 | 1.26 | 3.65 |
| CO₂ | 3.16 | 0.06 | 0.28 | 0.06 | 0.05 | 0.00 | 0.00 | 0.00 | 0.39 | 0.85 | 0.04 |
| N₂ | 84.66 | 0.16 | 0.77 | 0.16 | 0.05 | 0.00 | 0.00 | 0.00 | 1.03 | 2.32 | 0.01 |
| O₂ | 8.96 | 0.00 | 0.00 | 0.00 | 0.00 | 0.00 | 0.00 | 0.00 | 0.00 | 0.00 | 0.00 |

### Example 2

The example describes the use of mesitylene in the quenching stage. Excellent dissolution of a series of high-boiling secondary components is in this way effected in the quench and deposits downstream of the quench are prevented.

A process gas 2 having a temperature of 190° C., a pressure of 1.3 bar and the composition shown in Table 3 is provided from the ODH reactor 1. This gas stream is, in the quenching part 3, cooled to a temperature of 71° C. by means of a mesitylene/water recycle stream 4 having a phase ratio of 8:1 (mesitylene:water) and a temperature of 35° C. Here, a series of secondary components are dissolved out of the gas stream and the composition of the process gas stream changes to the concentrations shown for stream 6. The mass ratio of the recycle stream 4 to the process gas 2 and to the purge stream 4a is 1:0.1:0.01. The stream 4b comprises firstly the purge stream 8a (2nd quenching stage) and secondly a make-up stream of fresh mesitylene/water in a phase ratio of 5:2 (mesitylene:water).

In the second quenching stage 7, the gas stream 6 is cooled further to 54° C. by means of a further mesitylene recycle stream 8 which enters the quench at the top end at 35° C. The resulting gas stream 10 displays the decrease in concentration of secondary components shown in Table 4, while the mass ratio of stream 6 to 8 is 1:3.8 and a purge stream 8a making up 4.25% of stream 8 is taken off and fed to the first quenching part. The stream 8b consists of 100% of fresh mesitylene.

It has been observed that, in particular, the relatively nonvolatile materials and the materials present as solid under the process conditions in the quench are removed to a large extent from the process gas 2.

TABLE 3

| | Stream 2 |
|---|---|
| | [% by weight] |
| Butane | 4.13 |
| Isobutane | 1.08 |
| 1-Butene | 0.03 |
| cis-2-Butene | 0.70 |
| trans-2-Butene | 1.78 |
| 1,3-Butadiene | 9.96 |
| Water | 8.83 |
| Carbon dioxide | 1.19 |
| Ethene | 0.00 |
| 1-Vinylcyclohexene | 0.00 |
| Phthalic anhydride | 0.03 |
| Maleic anhydride | 0.13 |
| Benzoic acid | 0.02 |
| Benzaldehyde | 0.05 |
| Acetic acid | 0.05 |
| Furan | 0.00 |
| Methacrolein | 0.22 |
| 2-Butenal | 0.01 |
| Propionic acid | 0.06 |
| Maleic acid | 0.02 |
| Crotonic acid | 0.00 |
| Ethylbenzene | 0.01 |
| Styrene | 0.01 |
| Furanone | 0.00 |
| Fluorenone | 0.02 |
| Anthraquinone | 0.00 |
| Formaldehyde | 0.11 |
| Mesitylene | 0.00 |
| Carbon monoxide | 0.29 |
| Oxygen | 6.21 |
| Nitrogen | 64.37 |
| Argon | 0.69 |

TABLE 4

| | Removal % |
|---|---|
| 1-Vinylcyclohexene | 87.6296437 |
| Phthalic anhydride | 100 |
| Maleic anhydride | 100 |
| Benzoic acid | 100 |
| Benzaldehyde | 83.9332076 |
| Acetic acid | 100 |
| Methacrolein | 11.6162405 |
| Propionic acid | 100 |
| Maleic acid | 100 |
| Crotonic acid | 100 |
| Ethylbenzene | 57.5872721 |
| Styrene | 91.5361693 |
| Furanone | 100 |
| Fluorenone | 100 |
| Anthraquinone | 100 |
| Formaldehyde | 77.715195 |

1. A process for preparing butadiene from n-butenes, which comprises the steps:
A) provision of a feed gas stream a comprising n-butenes;
B) introduction of the feed gas stream a comprising n-butenes and an oxygen-comprising gas into at least one oxidative dehydrogenation zone and oxidative dehydrogenation of n-butenes to butadiene, giving a product gas stream b comprising butadiene, unreacted n-butenes, water vapor, oxygen, low-boiling hydrocarbons, high-boiling secondary components, possibly carbon oxides and possibly inert gases;
Ca) cooling of the product gas stream b by contacting with an organic solvent as coolant,
Cb) compression of the product gas stream b in at least one compression stage, giving at least one aqueous condensate stream c1 and a gas stream c2 comprising butadiene, n-butenes, water vapor, oxygen, low-boiling hydrocarbons, possibly carbon oxides and possibly inert gases;
D) separation of incondensable and low-boiling gas constituents comprising oxygen, low-boiling hydrocarbons, possibly carbon oxides and possibly inert gases as gas stream d2 from the gas stream c2 by absorption of the C4-hydrocarbons comprising butadiene and n-butenes in an absorption medium, giving an absorption medium stream loaded with C4-hydrocarbons and the gas stream d2, and subsequent desorption of the C4-hydrocarbons from the loaded absorption medium stream to give a C4-product gas stream d1,
E) separation of the C₄ product stream d1 by extractive distillation using a solvent selective for butadiene into a stream e1 comprising butadiene and the selective solvent and a stream e2 comprising n-butenes;
F) distillation of the stream e1 comprising butadiene and the selective solvent to give a stream consisting essentially of the selective solvent and a stream f2 comprising butadiene f1.

2. The process according to claim 1, wherein the solvent used in step Ca) is selected from among toluene, o-, m- and p-xylene and mixtures thereof.

3. The process according to claim 1, wherein the step Ca) is carried out in a plurality of stages.

4. The process according to claim 3, wherein the step Ca) is carried out in two stages Ca1) and Ca2).

5. The process according to claim 4, wherein at least part of the solvent which has passed through the second stage Ca2) is introduced as coolant into the first stage Ca1).

6. The process according to claim 1, wherein the stage Cb) comprises at least one compression stage Cba) and at least one cooling stage Cbb).

7. The process according to claim 6, wherein the gas compressed in the compression stage is brought into contact with a coolant in the at least one cooling stage.

8. The process according to claim 7, wherein the coolant in the cooling stage Cbb) comprises the same organic solvent used as coolant in stage Ca).

9. The process according to claim 8, wherein at least part of the coolant which has passed through the at least one cooling stage Cbb) is introduced as coolant into stage Ca).

10. The process according to claim 5, wherein the stage Cb) comprises a plurality of compression stages Cba1) to Cban) and cooling stages Cbb1) to Cbbn).

11. The process according to claim 1, wherein step D) comprises the steps Da) to Dc):
Da) absorption of the C₄-hydrocarbons comprising butadiene and n-butenes in a high-boiling absorption medium, giving an absorption medium stream loaded with C₄-hydrocarbons and the gas stream d2,
Db) removal of oxygen from the absorption medium stream loaded with C₄-hydrocarbons from step Da) by stripping with an incondensable gas stream and
Dc) desorption of the C₄-hydrocarbons from the loaded absorption medium stream, giving a C₄-product gas stream d1 which comprises less than 100 ppm of oxygen.

12. The process according to claim 10, wherein the high-boiling absorption medium used in step Da) is an aromatic hydrocarbon solvent.
